(19) United States
(12) Patent Application Publication
(10) Pub. No.: US 2004/0116462 A1
(43) Pub. Date: Jun. 17, 2004

(54) INDOLIZINE COMPOUNDS

(76) Inventors: Mitsunori Ono, Lexington, MA (US); Lijun Sun, Harvard, MA (US); Zhi Qiang Xia, Acton, MA (US); Hao Li, Brookline, MA (US); Shojun Chen, Bedford, MA (US); Masazumi Nagai, Lexington, MA (US); Rongzhen Lu, Cambridge, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/319,401

(22) Filed: Dec. 12, 2002

(51) Int. Cl. C07D 471/02

(52) U.S. Cl. 514/300; 546/138

(57) ABSTRACT

This invention relates to indolizine compounds having formula

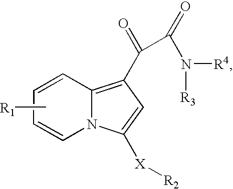

in which R₁ is H, lower alkyl, lower alkoxy, OH, F, Cl, Br, I, NO₂, or CN; R₂ is alkyl optionally substituted with lower alkoxy, OH, CN, F, Cl, Br, I, NO₂, NH₂, C(O)NH₂, CO₂H, or CO₂R′; or aryl optionally substituted with lower alkyl, lower alkoxy, OH, CN, F, Cl, Br, I, NO₂, NH₂, or C(O)NH₂, CO₂H, or CO₂R′; R₃ is H or lower alkyl; R₄ is N-oxy pyridyl or pyridyl optionally substituted with F, Cl, Br, or I; and X is C(R′R″), N(R′), O, S, S(O), S(O)₂, C(O), C(O)—N(R′), N(R′)—C(O), or deleted. Each of R′ and R″, independently, is H, or alkyl optionally substituted with lower alkoxy, OH, CN, F, Cl, Br, I, NO₂, NH₂, or C(O)NH₂.

### CROSS REFERENCE TO RELATED APPLICATIONS

This application claims the benefit of U.S. Utility application Ser. No. 10/244,088, filed Aug. 27, 2002, which, in turn, claims the benefit of U.S. Provisional Application Serial No. 60/322,020, filed Sep. 13, 2001.

### BACKGROUND

Recent studies have revealed that inhibition of the production or action of tumor necrosis factor alpha (TNFα) has therapeutic effects against inflammatory disorders such as multiple sclerosis, pulmonary fibrosis, atherosclerosis, and Crohn's disease. See Newton et al., J. Med. Chem. (1999) 42(13): 2295-2314. TNFα also plays an important role as a proinflammatory mediator in the development and progression of heart failure. See Mann, D. L., Circ. Res. (2002) 91:988-998. The activity of TNFα can be inhibited by antibodies. However, this immunotherapy can be expensive and inconvenient to treat chronic diseases because the antibodies are administered intravenously once or twice a month in a hospital. Also, antibodies, like most other proteins, tend to be unstable after administration.

Preclinic and clinic studies on phosphodiesterase 4 (PDE4) inhibitors have demonstrated that these agents may find utility in a wide range of inflammatory disorders, including asthma, chronic obstructive pulmonary disease, atopic dermatitis, rheumatoid arthritis, multiple sclerosis, and various neurological disorders. See Doherty, A. M., Current Opinion in Chemical Biology (1999) 3:466-473. No PDE4 inhibitors have been used as drugs to treat inflammatory diseases.

### SUMMARY

This invention is based on the discovery that certain indolizine compounds are effective in treating inflammatory disorders.

In one aspect, this invention features indolizine compounds of formula (I):

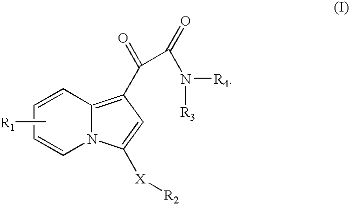

In the above formula, R₁ is H, lower alkyl, lower alkoxy, OH, F, Cl, Br, I, NO₂, or CN; R₂ is alkyl optionally substituted with lower alkoxy, OH, CN, F, Cl, Br, I, NO₂, NH₂, C(O)NH₂, CO₂H, or CO₂R′; or aryl optionally substituted with lower alkyl, lower alkoxy, OH, CN, F, Cl, Br, I, NO₂, NH₂, or C(O)NH₂, CO₂H, or CO₂R′; R₃ is H or lower alkyl; R₄ is N-oxy pyridyl or pyridyl optionally substituted with F, Cl, Br, or I; and X is C(R′R″), N(R′), O, S, S(O), S(O)₂, C(O), C(O)—N(R′), N(R′)—C(O), or deleted. Each of R′ and R″, independently, is H, or alkyl optionally substituted with lower alkoxy, OH, CN, F, Cl, Br, I, NO₂, NH₂, or C(O)NH₂.

Referring to formula (I), a subset of the compounds described above are those in which R₁ is H, OH, F, or Cl; R₂ is phenyl optionally p-substituted with lower alkoxy, OH, CN, F, Cl, Br, I, NO₂, NH₂, C(O)NH₂, CO₂H, or CO₂R′; R₃ is H; R₄ is N-oxy p-pyridyl optionally substituted with F, Cl, Br, or I, or p-pyridyl also optionally substituted with one or more halogens; and X is CH₂. Another subset of the compounds covered by formula (I) are those in which R₄ is o-pyridyl or m-pyridyl optionally substituted with F, Cl, Br, or I.

The term “alkyl” refers to both cyclic and acyclic, saturated and unsaturated non-aromatic C₁-C₁₀ hydrocarbon moieties, e.g., CH₃, CH═C₂H₅, or C₆H₁₁ (cyclic) and also includes those groups in which one or more carbon atoms are replaced with O, S, or N. The terms “lower alkyl” and “lower alkoxy” refer to C₁-C₄ alkyl and alkoxy, respectively. The term “aryl” refers to both hydrocarbon aryl moieties and heteroaryl moieties. Examples of hydrocarbon aryl moieties include phenyl, naphthyl, pyrenyl, anthryl, and phenanthryl. Examples of heteroaryl moieties include furyl, fluorenyl, pyrrolyl, thienyl, oxazolyl, imidazolyl, thiazolyl, pyridyl, pyrimidinyl, quinazolinyl, and indolyl.

Shown below are exemplary compounds, compounds 1-39, of this invention:

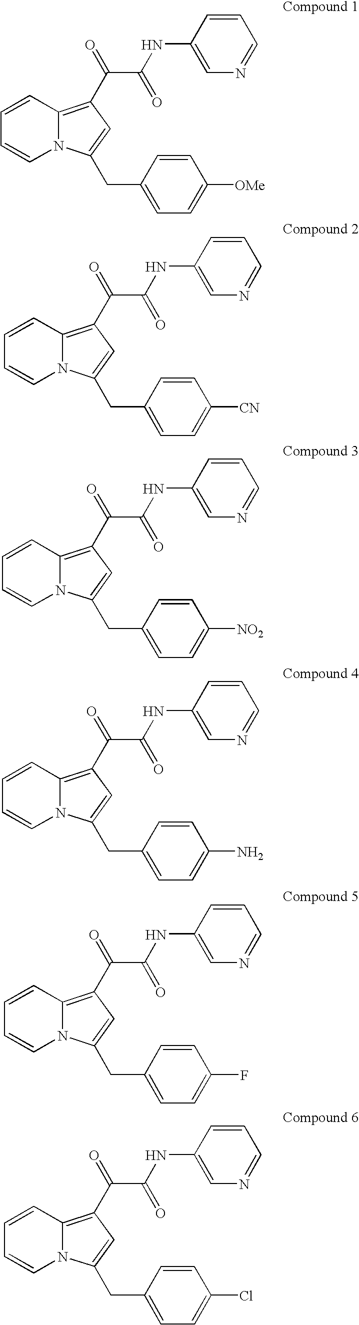

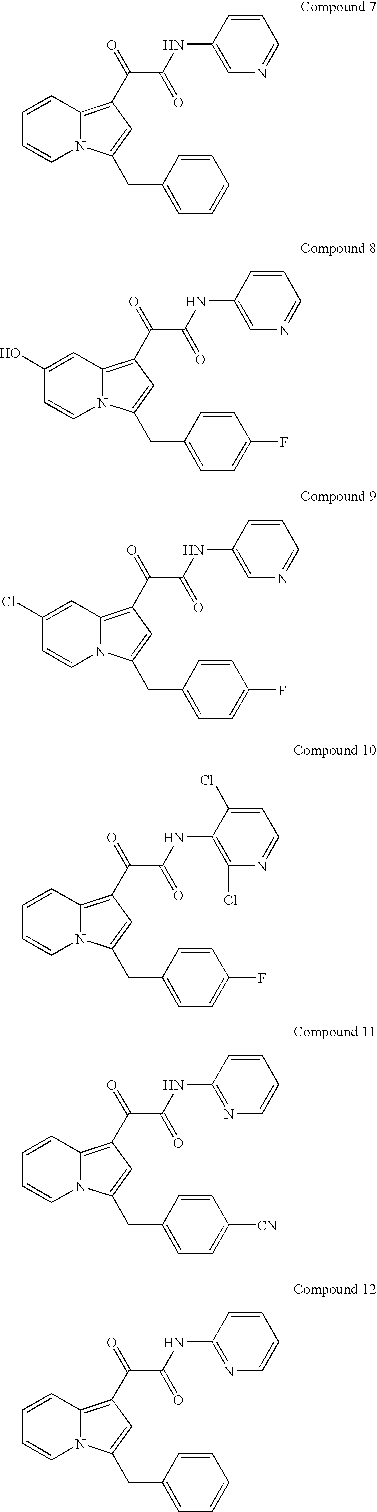

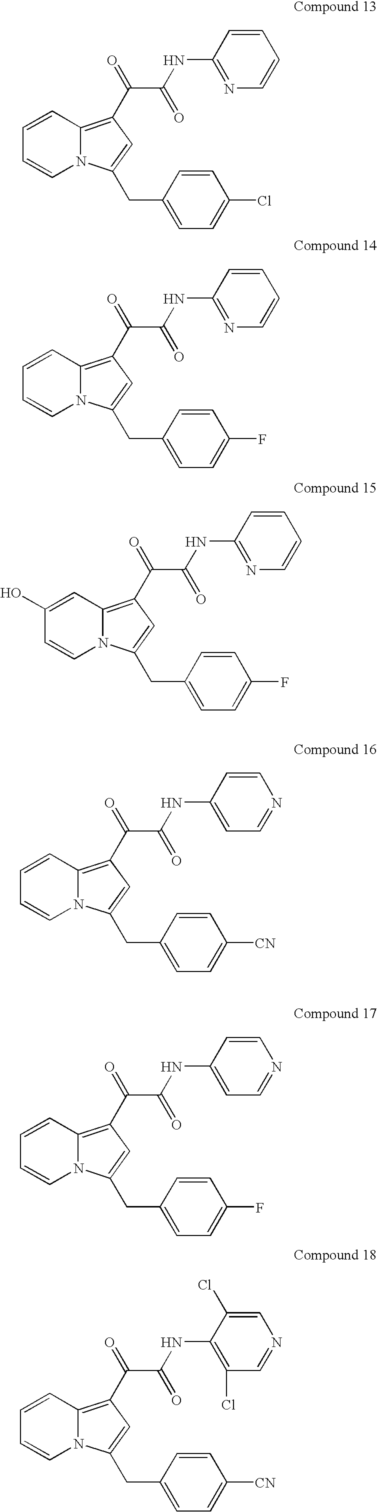

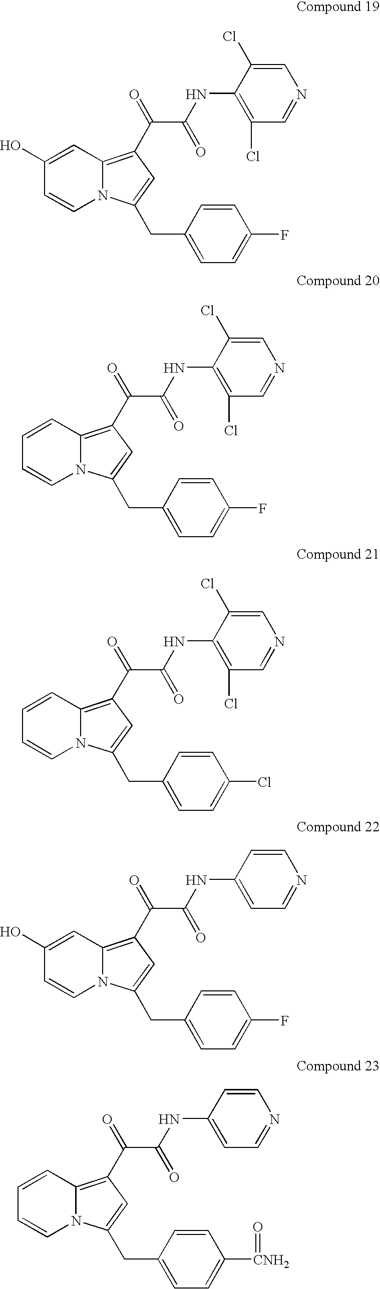

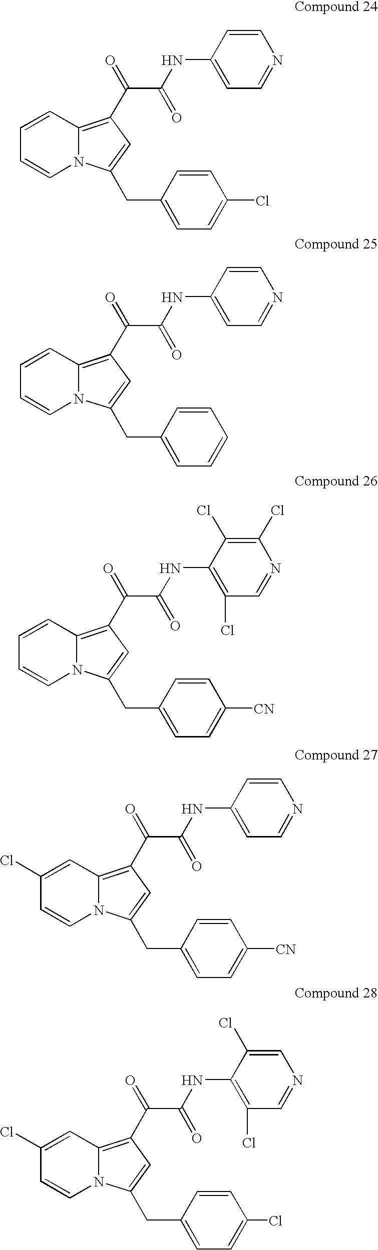

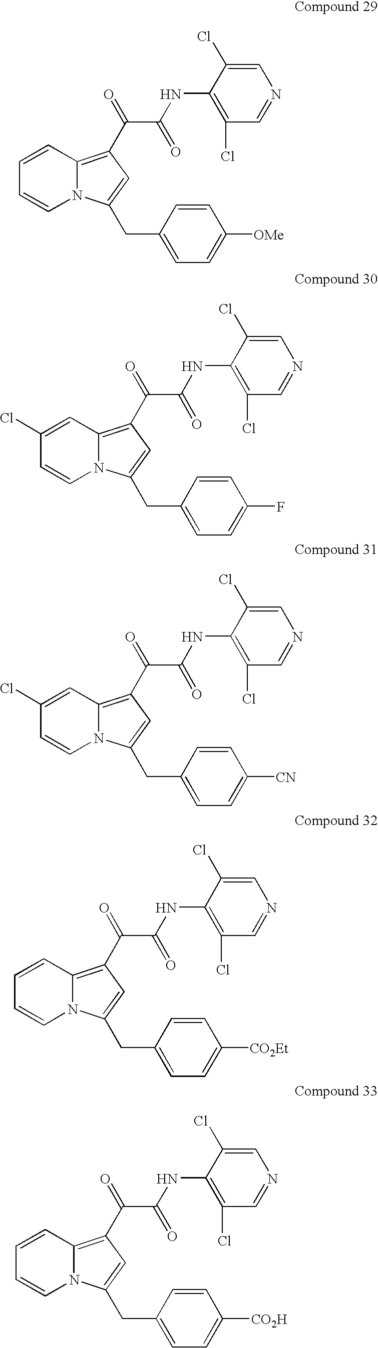

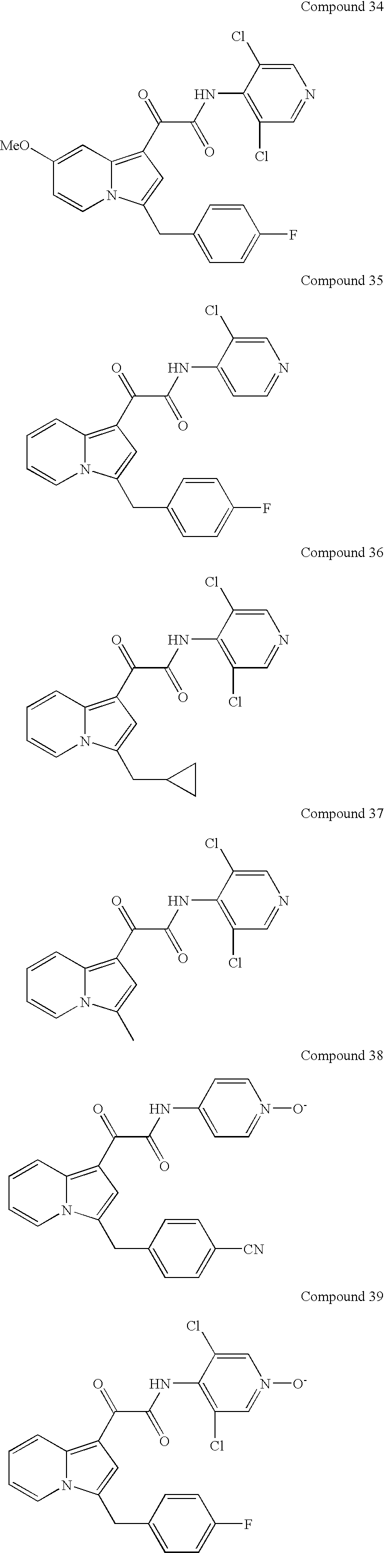

In another aspect, this invention features a pharmaceutical composition that contains an effective amount of at least one of the indolizine compounds described above and a pharmaceutically acceptable carrier.

In still another aspect, this invention features a method for treating inflammatory disorders, including inflammatory bowel disease (e.g., Crohn's disease), asthma, sepsis, stroke, heart failure, chronic obstructive pulmonary disease, allergic rhinitis, and autoimmune diseases (e.g., arthritis, multiple sclerosis, atherosclerosis, and psoriasis). The method includes administering to a subject in need thereof an effective amount of one or more of the above-described indolizine compounds. “Treatment of an inflammatory disorder” herein refers to administering a composition of the invention to a subject, who has an inflammatory disorder, a symptom of such a disorder or a predisposition towards such a disorder, with the purpose to cure, relieve, alter, affect, or prevent the inflammatory disorder, the symptom of it, or the predisposition towards it.

The indolizine compounds of this invention include the compounds themselves, as well as their salts and their prodrugs, if applicable. A salt, for example, can be formed between an anion and a positively charged substituent (e.g., amino) on an indolizine compound. Suitable anions include chloride, bromide, ioide, sulfate, nitrate, phosphate, citrate, methanesulfonate, trifluoroacetate, and acetate. Likewise, a salt can also be formed between a cation and a negatively charged substituent (e.g., carboxylate) on an indolizine compound of this invention. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active indolizine compounds.

Also within the scope of this invention is a composition containing one or more of the indolizine compounds described above for use in treating an inflammatory disorders, and the use of such a composition for the manufacture of a medicament for the just-mentioned use.

The details of one or more embodiments of the invention are set forth in the description below. Other features, objects, and advantages of the invention will be apparent from the following description and from the claims.

### DETAILED DESCRIPTION

The scheme below depicts the syntheses of a large number of indolizine compounds of this invention, i.e., compounds 1-39. Details of preparation of compounds 1-39 are described in Examples 1-39, respectively.

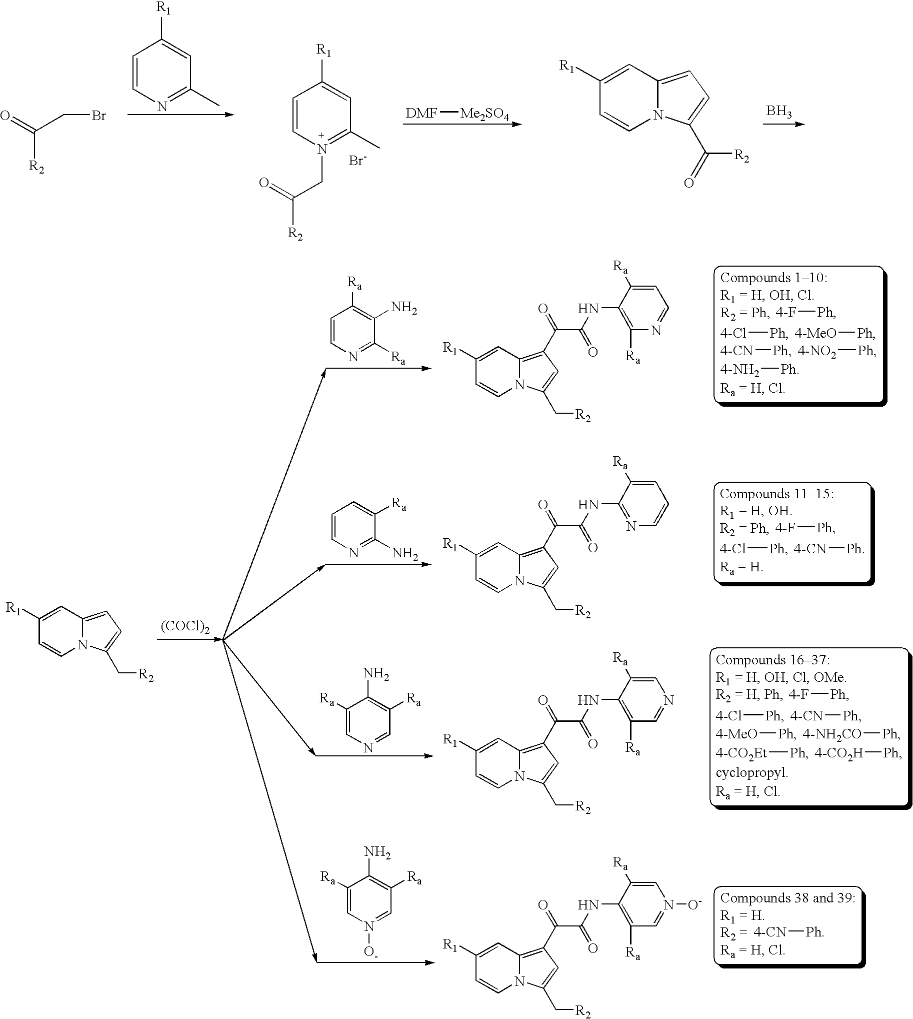

The indolizine compounds described above can be prepared by methods well known in the art, as well as by the synthetic routes disclosed herein. For example, one can react a 2-methylpyridine compound with a bromomethyl ketone compound to produce a pyridine salt. Treated with dimethyl sulfate, this pyridine salt forms an indolizine ring to give an indolizinyl ketone. This ketone can then be reduced to a 3-subsituted indolizine compound. A compound of this invention can be obtained by reacting the 3-substituted indolizine compound with 2-, 3-, or 4-aminopyridine or N-oxy 4-aminopyridine. Appropriate functional groups can be introduced into both the 2-methylpyridine compound and the aminopyridine compound. Any reactive groups on an indolizine intermediate can be protected prior to reacting the intermediate with an aminopyridine. For suitable protecting groups, see, e.g., Greene (1981) Protective Groups in Organic Synthesis, John Wiley & Sons, Inc., New York. An indolizine compound thus synthesized can be further purified by any conventional purification method, including without limitation, crystallization, flash column chromatography, solvating gas chromatography, or high performance liquid chromatography.

The indolizine compounds of the invention may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

Also within the scope of this invention is a pharmaceutical composition contains an effective amount of at least one indolizine compound of the present invention and a pharmaceutical acceptable carrier. Further, this invention covers a method of administering an effective amount of one or more of the indolizine compounds described in the summary section above to an inflammatory disorder patient. “An effective amount” refers to the amount of an active indolizine compound that is required to confer a therapeutic effect on the treated subject. Effective doses will vary, as recognized by those skilled in the art, depending on the types of diseases treated, route of administration, excipient usage, and the possibility of co-usage with other therapeutic treatment.

To practice the method of the present invention, a composition having one or more indolizine compound can be administered parenterally, orally, nasally, rectally, topically, or buccally. The term “parenteral” as used herein refers to subcutaneous, intracutaneous, intravenous, intrmuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions, and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having one or more active indolizine compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active indolizine compound. Examples of other carriers include colloidal silicon oxide, magnesium stearate, cellulose, sodium lauryl sulfate, and D&C Yellow #10.

The indolizine compounds of this invention can be preliminarily screened for their efficacy in treating inflammatory disorders by one or more of the following in vitro assays (See Examples 40 and 41 below) and in vivo assays (See Examples 42, 43, and 44 below). Other methods will also apparent to those of ordinary skill in the art.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### Preparation of Compound 1: 2-[3-(4-methoxy-benzyl)-indolizin-1-yl]-2-oxo-N-pyridin-3-yl-acetamide

Picoline (2.18 mL, 22 mmol) was added to a stirred solution of 2-bromo-1-(4-methoxyphenyl)-ethanone (2.5 g, 10.9 mmol) in 11 mL of acetonitrile at room temperature. The solution was continually stirred at room temperature for 2 hours. 15 mL of ethyl acetate was then added to the above solution. The resultant precipitate was collected by filtration and washed with ethyl acetate to give intermediate 1 as a white solid (2.4 g, 68%).

To a stirred suspension of intermediate 1 (2.4 g, 7.45 mmol) in 22 mL of dimethylformamide (DMF) was added 18 mL of DMF-Me₂SO₄ (obtained by stirring a mixture of 1 eq. DMF and 1 eq. Me₂SO₄ at 60-80° C. for 3 hours, followed by cooling to room temperature). Stirring was continued at room temperature for another 15 minutes. 31 mL of triethylamine was added to the above suspension, followed by stirring at 40-50° C. (reaction temperature) for 2 hours. After cooled to room temperature, the mixture was poured into 100 mL of ice water and was stirred for several hours. The resultant precipitate was collected, washed with water, and dried to give of intermediate 2 as an orange solid (1.4 g, 75%).

BH₃-THF (1M, 26 mL) was added to a solution of intermediate 2 (1.4 g, 11.2 mmol) in 33 mL of acetonitrile containing 0.5 mL of methanol. The resulting solution was stirred at 50° C. for 1 hour. The reaction mixture was cooled to ˜10° C. and quenched with 4 mL of ice water. 20 mL of ethyl acetate was added to the mixture, followed by drying with anhydrous Na₂SO₄. The solution was then decanted and evaporated under reduced pressure. The crude product was purified by solvating gas chromatography (SGC) using a gradient elution (hexane to 8:1 hexane/dichloromethane to 1:1 hexane/dichloromethane) to give intermediate 3 as an off-white solid (0.6 g, 43%).

A 10 mL dry ether solution containing intermediate 3 (0.39 g, 1.65 mmol) was slowly added to a stirred solution of oxalyl chloride (0.17 mL, 1.98 mmol) in 5 mL of dry ether at 0° C. After stirred at the same temperature for 30 minutes, the solution was concentrated to yield a solid and the resultant solid was re-dissolved in 5 mL of dry THF. A solution of 3-aminopyridine (0.37 g, 3.95 mmol) in 910 mL of dry THF was then added slowly to the above solution at 0° C. Stirring was continued at 0° C. for 1 hour and at room temperature for 2.5 hours. The volatiles were then removed under reduced pressure and the residue was dissolved in 30 mL of ethyl acetate. The ethyl acetate solution was washed successively with H₂O, saturated NaHCO₃, and brine. After dried with Na₂SO₄, the solvent of the solution was removed. The crude product was purified by SGC using a gradient elution (hexane to 2:1 hexane/ethyl acetate to 1:1 hexane/ethyl acetate) to give compound 1 as a yellow solid (0.3 g, 47%).

¹H NMR (CDCl₃) δ (ppm): 3.65 (s, 3H); 4.10 (s, 2H); 6.60-6.82 (m, 3H); 7.15 (q, J=7 Hz, 2H); 7.19-7.24 (m, 2H); 7.78 (d, J=7 Hz, 1H); 7.97 (s, 1H); 8.18-8.24 (m, 1H); 8.32 (dd, J=1.5 Hz, 5 Hz, 1H); 8.52 (d, J=9 Hz, 1H); 8.74 (d, J=2.4 Hz, 1H); 9.55 (s, 1H).

ESMS calculated for (C₂₃H₁₉N₃O₃): 385.1; found: 386.1 (M+H)⁺.

### EXAMPLE 2

### Preparation of Compound 2: 2-[3-(4-cyano-benzyl)-indolizin-1-yl]-2-oxo-N-pyridin-3-yl-acetamide

Compound 2 was prepared in a manner similar to that described in Example 1.

¹H-NMR (CDCl₃) δ (ppm): 9.52 (s, 1H); 8.81 (d, J=4.8 Hz, 1H); 8.66 (d, J=12 Hz, 1H); 8.45-8.26 (m, 2H); 8.13 (s, 1H); 7.81 (d, J=12 Hz, 1H); 7.64-7.30 (m, 6H); 6.96 (m, 1H); 4.26 (s, 2H).

ESMS calculated for (C₂₃H₁₆N₄O₂): 380.13; Found: 381.2 (M+H)⁺.

### EXAMPLE 3

### Preparation of Compound 3: 2-[3-(4-nitro-benzyl)-indolizin-1-yl]-2-oxo-N-pyridin-3-yl-acetamide

Compound 3 was prepared in a manner similar to that described in Example 1.

¹H NMR (CDCl₃) δ (ppm): 4.25 (s, 2H); 6.83 (t, J=7 Hz, 1H); 7.14-7.32 (m, 4H); 7.71 (d, J=7 Hz, 1H); 7.97 (s, 1H); 8.03 (d, J=8 Hz, 2H); 8.17 (dt, J=8 Hz, 1.5 Hz, 1H); 8.28 (d, J=4.8 Hz, 1H); 8.48 (dd, J=9 Hz, 1.2 Hz, 1H); 8.74 (s, 1H); 9.59 (s, 1H).

ESMS calculated for (C₂₂H₁₆N₄O₄): 400.1; found: 401.1 (M+H)⁺.

### EXAMPLE 4

### Preparation of Compound 4: 2-[3-(4-amino-benzyl)-indolizin-1-yl]-2-oxo-N-pyridin-3-yl-acetamide

Compound 4 was prepared in a manner similar to that described in Example 1.

¹H NMR (CDCl₃) δ (ppm): 3.65 (bs, 2H); 4.18 (s, 2H); 6.63 (d, J=9 Hz, 1H); 6.9 (t, J=7 Hz, 1H); 6.98 (d, J=7 Hz, 2H); 7.28-7.39 (m, 2H); 7.90 (d, J=7 Hz, 1H); 8.06 (s, 1H); 8.28-8.34 (m, 1H); 8.41 (dd, J=5 Hz, 1.5 Hz, 1H); 8.63 (d, J=9 Hz, 1H); 8.83 (d, J=2.4 Hz, 1H); 9.62 (s, 1H).

ESMS calculated for (C₂₂H₁₈N₄O₂): 370.1; found: 371.1 (M+H)⁺.

### EXAMPLE 5

### Preparation of Compound 5: 2-[3-(4-fluoro-benzyl)-indolizin-1-yl]-2-oxo-N-pyridin-3-yl-acetamide

Compound 5 was prepared in a manner similar to that described in Example 1.

¹H NMR (CDCl₃) δ (ppm): 9.53 (s, 1H); 8.81 (d, J=3.0 Hz, 1H); 8.66 (d, J=9.0 Hz, 1H); 8.40 (d, J=6.0 Hz, 2H); 8.30 (s, 1H); 7.85 (d, J=9.0 Hz, 1H); 7.35 (m, 3H); 7.96 (m, 3H); 4.24 (s, 2H).

ESMS calculated (C₂₂H₁₆FN₃O₂): 373.1; found: 374.1 (M+H)⁺

### EXAMPLE 6

### Preparation of Compound 6: 2-[3-(4-chloro-benzyl)-indolizin-1-yl]-2-oxo-N-pyridin-3-yl-acetamide

Compound 6 was prepared in a manner similar to that described in Example 1.

¹H NMR (CDCl₃) δ (ppm): 9.53 (s, 1H); 8.81 (d, J=3.0 Hz, 1H); 8.66 (d, J=9.0 Hz, 1H); 8.42 (d, J=6.0 Hz, 1H); 8.30 (m, 1H); 8.11 (s, 1H); 7.83 (d, J=6.0 Hz, 1H); 7.36 (m, 3H); 7.15 (d, J=9.0 Hz, 2H); 6.93 (m, 1H); 4.24 (s, 2H).

ESMS calculated (C₂₂H₁₆ClN₃O₂): 389.1; found: 390.1 (M+H)⁺

### EXAMPLE 7

### Preparation of Compound 7: 2-(3-benzyl-indolizin-1-yl)-2-oxo-N-pyridin-3-yl-acetamide.

Compound 7 was prepared in a manner similar to that described in Example 1.

¹H NMR (CDCl₃) δ (ppm): 4.20 (s, 2H); 6.82 (t, J=7 Hz, 1H); 7.11-7.32 (m, 7H); 7.80 (d, J=7 Hz, 1H); 8.04 (s, 1H); 8.22-8.27 (m, 1H); 8.34 (dd, J=1.4 Hz, 5 Hz, 1H); 8.56 (d, J=9 Hz, 1H); 8.74 (d, J=3 Hz, 1H); 9.49 (s, 1H).

ESMS calculated for (C₂₂H₁₇N₃O₂): 355.1; found: 356.1 (M+H)⁺.

### EXAMPLE 8

### Preparation of Compound 8: 2-[3-(4-fluoro-benzyl)-7-hydroxy-indolizin-1-yl]-2-oxo-N-pyridin-3-yl-acetamide

3.2 g (60%, 80 mmol) of NaH was added to a 130 mL DMF solution containing 8.88 g (82 mmol) of benzyl alcohol at 0° C. The solution was stirred at room temperature for 2 hours. 10 g (78 mmol) of 4-chloropicoline was added to the above solution at room temperature, followed by stirring the solution for another 3 hours at 100° C. 200 mL of ice water was added to the above solution to yield a precipitate. The resultant precipitate was collected, washed with water, and dried to give 13.8 g (79%) of 4-benzyloxy-2-methyl-pyridine.

Compound 8 with a benzyl protected hydroxy(2-[3-(4-fluoro-benzyl)-7-benzyloxy-indolizin-1-yl]-2-oxo-N-pyridin-3-yl-acetamide) was prepared in a manner similar to that described in Example 1 by using 4-benzyloxy-2-methyl-pyridine was used as a starting material.

The protecting benzyl group was removed by using a well-known hydrogenation reduction to give compound 8.

¹H-NMR (CD₃SOCD₃) δ (ppm): 10.72 (s, 1H); 9.01 (s, 1H); 8.32 (d, J=6 Hz, 1H); 8.26 (d, J=12 Hz, 2H); 7.89 (s, 1H); 7.42-7.10 (m, 6H); 7.64-7.30 (m, 6H); 4.22 (s, 2H).

ESMS calculated for (C₂₂H₁₆FN₃O₃): 389.12; found: 390.1 (M+H)⁺.

### EXAMPLE 9

### Preparation of Compound 9: 2-[7-Chloro-3-(4-fluoro-benzyl)-indolizin-1-yl]-2-oxo-N-pyridin-3-yl-acetamide

Compound 9 was prepared in a manner similar to that described in Example 1.

¹H-NMR (CDCl₃, 300 MHz) δ (ppm): 9.46 (s, 1H); 8.82 (d, J=2.4 Hz, 1H); 8.68 (d, J=2.1 Hz, 1H); 8.42 (d, J=5.1 Hz, 1H); 8.31-8.23 (m, 1H); 8.09 (s, 1H); 7.74 (d, J=7.5 Hz, 1H); 7.34 (dd, J=5.1 Hz, 8.1 Hz, 1H); 7.18-7.14 (m, 2H); 7.04-6.98 (m, 2H); 6.89 (dd, J=2.4 Hz, 7.5 Hz, 1H); 4.22 (s, 2H);

ESMS: calculated for (C₂₂H₁₅ClFN₃O₂): 407.82; found: 408.0 (M+H)⁺.

### EXAMPLE 10

### Preparation of Compound 10: 2-[3-(4-fluoro-benzyl)-indolizin-1-yl]-2-oxo-N-(2,4-dichloro-pyridin-3-yl)-acetamide

Compound 10 was prepared in a manner similar to that described in Example 1.

¹H-NMR (CDCl₃) δ (ppm): 9.44 (s, 1H); 8.60 (d, J=12 Hz, 1H); 8.24 (d, J=10 Hz, 1H), 7.96 (s, 1H); 7.83 (d, J=12 Hz, 1H); 7.39 (d, J=10 Hz, 1H); 7.38-6.89 (m, 6H); 4.21 (s, 2H).

ESMS calculated for (C₂₂H₁₄Cl₂FN₃O₂): 441.04; found: 442.0 (M+H)⁺.

### EXAMPLE 11

### Preparation of Compound 11: 2-[3-(4-cyano-benzyl)-indolizin-1-yl]-2-oxo-N-pyridin-2-yl-acetamide

Compound 11 was prepared in a manner similar to that described in Example 1.

¹H-NMR (CDCl₃) δ (ppm): 9.84 (s, 1H); 8.68 (d, J=12 Hz, 1H); 8.41-8.28 (m, 2H); 8.08 (s, 1H); 8.13 (s, 1H); 7.81-7.73 (m, 2H); 7.61 (d, J=12.5 Hz, 2H); 7.41-7.32 (m, 3H); 7.13-6.84 (m, 2H); 4.36 (s, 2H).

ESMS calculated for (C₂₃H₁₆N₄O₂): 380.13; Found: 381.2 (M+H)⁺.

### EXAMPLE 12

### Preparation of Compound 12: 2-(3-benzyl-indolizin-1-yl)-2-oxo-N-pyridin-2-yl-acetamide

Compound 12 was prepared in a manner similar to that described in Example 1.

¹H NMR (CDCl₃) δ (ppm): 4.24 (s, 2H); 6.92 (t, J=7 Hz, 1H); 7.20-7.41 (m, 7H); 7.68 (m, 1H); 7.89 (d, J=7 Hz, 1H); 8.10 (s, 1H); 8.55-8.61 (m, 1H); 8.65 (d, J=9 Hz, 1H); 9.60 (s, 1H).

ESMS calculated for (C₂₂H₁₇N₃O₂): 355.1; found: 356.1 (M+H)⁺.

### EXAMPLE 13

### Preparation of Compound 13: 2-[3-(4-chloro-benzyl)-indolizin-1-yl]-2-oxo-N-pyridin-2-yl-acetamide

Compound 13 was prepared in a manner similar to that described in Example 1.

¹H NMR (CDCl₃) δ (ppm): 9.89 (s, 1H); 8.81 (m, 1H); 8.38 (m, 2H); 8.07 (s, 1H); 7.78 (m, 2H); 7.36 (m, 3H); 7.08 (m, 2H); 6.89 (m, 1H); 4.23 (s, 2H).

ESMS calculated (C₂₂H₁₆ClN₃O₂): 389.1; found: 390.1 (M+H)⁺.

### EXAMPLE 14

### Preparation of Compound 42: 2-[3-(4-fluoro-benzyl)-indolizin-1-yl]-2-oxo-N-pyridin-2-yl-acetamide

Compound 12 was prepared in a manner similar to that described in Example 1.

¹H NMR (CDCl₃) δ (ppm): 9.77 (s, 1H); 8.66 (d, J=9.0 Hz, 1H); 8.40 (m, 2H); 8.06 (s, 1H); 7.81 (m, 3H); 7.35 (m, 1H); 7.26 (m, 4H); 4.24 (s, 2H).

ESMS calculated (C₂₂H₁₆FN₃O₂): 373.1; found: 374.1 (M+H)⁺.

### EXAMPLE 15

### Preparation of Compound 15: 2-[3-(4-fluoro-benzyl)-7-hydroxy-indolizin-1-yl]-2-oxo-N-pyridin-2-yl-acetamide

Compound 15 was prepared in a manner similar to that described in Example 8.

¹H-NMR (CD₃COCD₃) δ (ppm): 9.93 (s, 1H); 8.30-8.39 (m, 2H); 8.12-8.02 (m, 2H); 7.90-7.76 (m, 2H); 7.38-7.28 (m, 2H); 7.18-7.02 (m, 3H); 6.76-7.70 (m, 1H); 4.22 (s, 2H).

ESMS calculated for (C₂₂H₁₆FN₃O₃): 389.12; found: 390.1 (M+H)⁺.

### EXAMPLE 16

### Preparation of Compound 16: 2-[3-(4-cyano-benzyl)-indolizin-1-yl]-2-oxo-N-pyridin-4-yl-acetamide

Compound 16 was prepared in a manner similar to that described in Example 1.

¹H NMR (CDCl₃) 6 (ppm): 4.36 (s, 2H); 6.95 (t, J=3.8 Hz, 1H); 7.3-7.5 (m, 3H); 7.6-7.7 (m, 4H); 7.80 (d, J=3.9 Hz, 1H); 8.05 (s, 1H); 8.5-8.7 (m, 3H); 9.60 (s, 1H).

ESMS calculated (C₂₃H₁₆N₄O₂): 380.13; found: 381.1 (M+H)⁺.

### EXAMPLE 17

### Preparation of Compound 17: 2-[3-(4-fluoro-benzyl)-indolizin-1-yl]-2-oxo-N-pyridin-4-yl-acetamide

Compound 17 was prepared in a manner similar to that described in Example 1.

¹H NMR (DMSO-d₆) δ (ppm): 11.01 (s, 1H); 8.49 (d, J=6.3 Hz, 2H); 8.47-8.39 (m, 2H); 7.81 (d, J=6.3 Hz, 2H); 7.46-7.53 (m, 2H); 7.31-7.36 (m, 2H); 7.12-7.18 (m, 3H); 4.31 (s, 2H).

ESMS calculated (C₂₂H₁₆FN₃O₂): 373.38; found 374.4 (M+H)⁺.

### EXAMPLE 18

### Preparation of Compound 18: 2-[3-(4-cyano-benzyl)-indolizin-1-yl]-2-oxo-N-(3,5-dichloro-pyridin-4-yl)-acetamide

Compound 18 was prepared in a manner similar to that described in Example 1.

¹H-NMR (CDCl₃) δ (ppm): 9.48 (s, 1H); 8.66 (d, J=12 Hz, 1H); 8.59 (s, 2H); 8.03 (s, 1H); 7.81 (d, J=10, 1H); 7.62 (d, J=11.5 Hz, 2H); 7.41 (m, 1H); 7.32 (d, J=11.5 Hz, 1H); 6.95 (m, 1H); 4.32 (s, 2H).

ESMS calculated for (C₂₃H₁₄Cl₂N₄O₂): 448.05; found: 449.1 (M+H)⁺.

### EXAMPLE 19

### Preparation of Compound 19: 2-[3-(4-fluoro-benzyl)-7-hydroxy-indolizin-1-yl]-2-oxo-N-(3,5-dichloro-pyridin-4-yl)-acetamide

Compound 19 was prepared in a manner similar to that described in Example 8.

¹H-NMR (CD₃COCD₃) δ (ppm): 9.98 (s, 1H); 8.62 (s, 2H); 8.19 (d, J=11 Hz, 1H); 8.03 (s, 1H); 7.64 (s, 1H); 7.38-6.77 (m, 5H); 4.29 (s, 2H).

ESMS calculated for (C₂₂H₁₄Cl₂FN₃O₃): 457.04; found: 458.0 (M+H)⁺.

### EXAMPLE 20

### Preparation of Compound 20: 2-[3-(4-fluoro-benzyl)-indolizin-1-yl]-2-oxo-N-(3,5-dichloro-pyridin-4-yl)-acetamide

Compound 20 was prepared in a manner similar to that described in Example 1.

¹H-NMR (CDCl₃) δ (ppm): 9.49 (s, 1H); 8.63 (d, J=12 Hz, 1H); 8.59 (s, 2H); 8.01 (s, 1H); 7.84 (d, J=11 Hz, 1H); 7.43-6.92 (m, 6H); 4.21 (s, 2H).

ESMS calculated for (C₂₂H₁₄Cl₂FN₃O₂): 441.04; found: 440.0 (M−H)⁻.

### EXAMPLE 21

### Preparation of Compound 21: 2-[3-(4-chloro-benzyl)-indolizin-1-yl]-N-(3,5-dichloro-pyridin-4-yl)-2-oxo-acetamide

Compound 21 was prepared in a manner similar to that described in Example 1.

¹H NMR (CDCl₃) δ (ppm): 9.49 (s, 1H); 8.64 (m, 3H); 8.03 (s, 1H); 7.84 (d, J=9.0 Hz, 1H); 7.39 (m, 1H); 7.28 (m, 3H); 7.14 (d, J=9.0 Hz, 2H); 6.95 (m, 1H); 4.22 (s, 2H).

ESMS calculated (C₂₂H₁₆Cl₂N₃O₂): 457.0; found: 458.0 (M+H)⁺.

### EXAMPLE 22

### Preparation of Compound 22: 2-[3-(4-fluoro-benzyl)-7-hydroxy-indolizin-1-yl]-2-oxo-N-pyridin-4-yl-acetamide

Compound 22 was prepared in a manner similar to that described in Example 8.

¹H-NMR (CD₃SOCD₃) δ (ppm); 10.93 (s, 1H); 10.85 (s, 1H); 8.46 (d, J=4.5 Hz, 2H); 8.23 (d, J=9 Hz, 1H); 7.82 (s, 1H), 7.78 (d, J=4.5 Hz, 2H); 7.36-7.10 (m, 5H); 4.21 (s, 2H).

ESMS calculated for (C₂₂H₁₆FN₃O₃): 389.12; found: 390.1 (M+H)⁺.

### EXAMPLE 23

### Preparation of Compound 23: 4-[1-(pyridin-4-yl-aminooxalyl)-indolizin-3-yl-methyl]-benzamide

Compound 23 was prepared in a manner similar to that described in Example 1.

¹H NMR (DMSO-d₆) δ (ppm): 4.40 (s, 2H); 7.18 (t, J=6.9 Hz, 1H); 7.30-7.56 (m, 5H); 7.80-7.86 (m, 3H); 7.90-7.96 (br, 2H); 8.38-8.52 (m, 4H); 11.00 (s, 1H).

ESMS calculated for (C₂₃H₁₈N₄O₃): 398.1; found: 399.1 (M+H)⁺.

### EXAMPLE 24

### Preparation of Compound 24: 2-[3-(4-chloro-benzyl)-indolizin-1-yl]-2-oxo-N-pyridin-4-yl-acetamide

Compound 24 was prepared in a manner similar to that described in Example 1.

¹H NMR (CDCl₃) δ (ppm): 9.59 (s, 1H); 8.65 (d, J=9.0 Hz, 1H); 8.58 (d, J=6.0 Hz, 2H); 8.09 (s, 1H); 7.84 (d, J=6.0 Hz, 1H); 7.66 (m, 2H); 7.40 (m, 1H); 7.38 (m, 1H); 7.15 (d, J=6.0 Hz, 2H); 6.96 (m, 1H); 4.24 (s, 2H).

ESMS calculated (C₂₂H₁₆ClN₃O₂): 389.1; found: 390.1 (M+H)⁺.

### EXAMPLE 25

### Preparation of Compound 25: 2-(3-benzyl-indolizin-1-yl)-2-oxo-N-pyridin-4-yl-acetamide.

Compound 25 was prepared in a manner similar to that described in Example 1.

¹H NMR (CDCl₃) δ (ppm): 4.20 (s, 2H); 6.80 (t, J=7 Hz, 1H); 6.95-7.40 (m, 1H); 7.12-7.30 (m, 6H); 7.68 (t, J=8 Hz, 1H); 7.78 (d, J=7 Hz, 1H); 8.02 (s, 1H); 8.26-8.34 (m, 2H); 8.60 (d, J=9 Hz, 1H); 9.82 (s, 1H).

ESMS calculated for (C₂₂H₁₇N₃O₂): 355.1; found: 356.1 (M+H)⁺.

### EXAMPLE 26

### Preparation of Compound 26: 2-[3-(4-cyano-benzyl)-indolizin-1-yl]-2-oxo-N-(2,3,5-trichloro-pyridin-4-yl)-acetamide.

Compound 26 was prepared in a manner similar to that described in Example 1.

¹H-NMR (CDCl₃) δ (ppm): 9.57 (s, 1H); 8.64 (d, J=12 Hz, 1H); 8.41 (s, 1H); 8.07 (s, 1H); 7.80 (d, J=12 Hz, 1H); 7.61 (d, J=12.5 Hz, 2H); 7.42 (m, 1H); 7.31 (d, J=12.5 Hz, 2H); 6.98 (m, 1H); 4.32 (s, 2H).

ESMS calculated for (C₂₃H₁₃Cl₃N₄O₂): 482.01; found: 483.1.1 (M+H)⁺.

### EXAMPLE 27

### Preparation of Compound 27: 2-[7-chloro-3-(4-cyano-benzyl)-indolizin-1-yl]-2-oxo-N-pyridin-4-yl-acetamide

Compound 27 was prepared in a similar manner as described in Example 1.

¹H NMR (CDCl₃) δ (ppm): 4.32 (s, 2H); 6.92 (dd, J=7 Hz, 2 Hz, 1H); 7.20-7.35 (m, 3H); 7.55-7.72 (m, 4H); 8.09 (s, 1H); 8.59 (d, J=6 Hz, 2H); 8.68 (d, J=2 Hz, 1H); 9.52 (s, 1H).

ESMS calculated for (C₂₃H₁₅ClN₄O₂): 414.1; found: 415.1 (M+H)⁺.

### EXAMPLE 28

### Preparation of Compound 28: 2-[7-chloro-3-(4-chloro-benzyl)-indolizin-1-yl]-2-oxo-N-(3,5-dichloro-pyridin-4-yl)-acetamide

Compound 28 was prepared in a manner similar to that described in Example 1.

¹H NMR (300 MHz, CDCl₃) δ (ppm): 9.49 (s, 1H); 8.65 (d, J=2.4 Hz, 1H); 8.57 (s, 2H); 8.00 (s, 1H); 7.72 (d, J=7.2 Hz, 1H); 7.26 (d, J=8.4 Hz, 2H); 7.17 (d, J=8.4 Hz, 2H); 6.89 (dd, J=7.2 Hz, 2.4 Hz, 1H); 4.19 (s, 2H).

ESMS calculated for (C₂₂H₁₄Cl₄N₃O₂): 490.98; found: 492.1 (M+H)⁺.

### EXAMPLE 29

### Preparation of Compound 29: 2-[3-(4-methoxy-benzyl)-indolizin-1-yl]-2-oxo-N-(3,5-dichloro-pyridin-4-yl)-acetamide

Compound 29 was prepared in a manner similar to that described in Example 1.

¹H NMR (CDCl₃) δ (ppm): 9.5 (s, 1H); 8.66 (d, J=9.0 Hz, 1H); 8.58 (s, 2H); 8.01 (s, 1H); 7.89 (d, J=6.9 Hz, 1H); 7.39 (t, J=7.8 Hz, 1H); 7.12 (d, J=8.7 Hz, 2H); 6.93 (t, J=6.9 Hz, 1H); 6.84 (d, J=8.7 Hz, 2H); 4.19 (s, 2H); 3.78 (s, 3H);

ESMS calculated for (C₂₃H₁₇Cl₂N₃O₃): 453.06; found: 476.1 (M+Na)⁺.

### EXAMPLE 30

### Preparation of Compound 30: 2-[7-chloro-3-(4-fluoro-benzyl)-indolizin-1-yl]-2-oxo-N-(3,5-dichloro-pyridin-4-yl)-acetamide

Compound 30 was prepared in a manner similar to that described in Example 1.

¹H-NMR (CDCl₃, 300 MHz) δ (ppm): 9.42 (s, 1H); 8.69 (d, J=2.1 Hz, 1H); 8.58 (s, 2H); 8.01 (s, 1H); 7.75 (d, J=7.2 Hz, 1H); 7.17-7.13 (m, 2H); 7.03-6.97 (m, 2H); 6.90 (dd, J=2.1 Hz, 7.2 Hz, 1H); 4.20 (s, 2H).

ESMS calculated for (C₂₂H₁₃Cl₃FN₃O₂): 476.71 found: 500.0 (M+Na)⁺.

### EXAMPLE 31

### Preparation of Compound 31: 2-[7-chloro-3-(4-cyano-benzyl)-indolizin-1-yl]-2-oxo-N-(3,5-dichloro-pyridin-4-yl)-acetamide

Compound 31 was prepared in a manner similar to that described in Example 1.

¹H NMR (300 MHz, CDCl₃) δ (ppm): 9.51 (s, 1H); 8.63 (d, J=2.4 Hz, 1H); 8.55 (s, 2H); 8.00 (s, 1H); 7.71 (d, J=7.2 Hz, 1H); 7.58 (d, J=8.1 Hz, 2H); 7.30 (d, J=8.1 Hz, 2H); 6.90 (dd, J=7.2 Hz, 2.4 Hz, 1H); 4.29 (s, 2H);

ESMS calculated for (C₂₃H₁₄Cl₃N₄O₂): 483.0; found: 484.0 (M+H)⁺.

### EXAMPLE 32

### Preparation of Compound 32: 4-[1-(3,5-dichloro-pyridin-4-yl-aminooxalyl)indolizin-3-yl-methyl]-benzoic acid ethyl ester

Compound 32 was prepared in a manner similar to that described in Example 1.

H-NMR (CDCl₃) δ (ppm): 9.47 (s, 1H); 8.67 (d, J=8.7 Hz, 1H); 8.57 (s, 1H); 8.06 (s, 1H); 7.98 (d, J=8.1 Hz, 2H); 7.82 (d, J=7.2 Hz, 1H); 7.43-737 (m, 1H); 7.26 (s, 2H); 6.95-6.93 (m, 1H); 4.34 (q, J=7.2 Hz, 14.4 Hz, 2H); 4.31 (s, 2H), 1.37 (t, J=7.2 Hz, 3H).

ESMS calculated for (C₂₅H₁₉Cl₂N₃O₄): 495.08; found: 494.2 (M−H)⁻.

### EXAMPLE 33

### Preparation of Compound 33: 4-[1-(3,5-dichloro-pyridin-4-yl-aminooxalyl)-indolizin-3-yl-methyl]-benzoic Acid

Compound 33 was prepared in a manner similar to that described in Example 1.

¹H-NMR (CD₃OD) δ (ppm): 8.62-8.55 (m, 3H); 8.16 (d, J=6.0 Hz, 1H); 7.90-7.88 (m, 2H); 7.67 (s, 1H); 7.48-7.42 (m, 1H); 7.25 (s, 2H); 7.06-6.98 (m, 1H); 4.34 (s, 2H).

ESMS calculated for (C₂₃H₁₅Cl₂N₃O₄): 467.04; found: 468.0 (M+H)⁺.

### EXAMPLE 34

### Preparation of Compound 34: 2-[3-(4-fluoro-benzyl)-7-methoxy-indolizin-1-yl]-2-oxo-N-(3,5-dichloro-pyridin-4-yl)-acetamide

Compound 34 was prepared in a manner similar to that described in Example 1.

¹H-NMR (CDCl₃, 300 MHz) δ (ppm): 9.55 (s, 1H); 8.57 (s, 2H); 8.09 (d, J=2.7 Hz, 1H); 7.88 (s, 1H); 7.69 (dd, J=0.3 Hz, 7.2 Hz, 1H); 7.19-7.14 (m 2H); 7.01-6.95 (m, 2H); 6.63 (dd, J=2.7 Hz, 7.2 Hz, 1H); 4.16 (s, 2H); 3.97 (s, 3H).

ESMS calculated for (C₂₃H₁₆Cl₂FN₃O₃) (M+): 471.60; found: 494.0 (M+Na)⁺.

### EXAMPLE 35

### Preparation of Compound 35: 2-[3-(4-fluoro-benzyl)-indolizin-1-yl]-2-oxo-N-(3-chloro-pyridin-4-yl)-acetamide

Compound 35 was prepared in a manner similar to that described in Example 1.

¹H-NMR (CD₃ COCD₃) δ (ppm): 10.25 (s, 1H); 8.67-8.61 (m, 2H); 8.52 (s, 2H); 8.37 (d, J=10 Hz, 1H); 7.94 (s, 1H); 7.60-7.09 (m, 6H); 4.21 (s, 2H).

ESMS calculated for (C₂₂H₁₅ClFN₃O₂): 407.08; found: 408.0 (M+H)⁺.

### EXAMPLE 36

### Preparation of Compound 36: 2-(3-cyclopropylmethyl-indolizin-1-yl)-N-(3,5-dichloro-pyridin-4-yl)-2-oxo-acetamide

Compound 36 was prepared in a manner similar to that described in Example 1.

¹H-NMR (CDCl₃) δ (ppm): 9.50 (s, 1H); 8.67-85.7 (m, 3H); 8.06-8.03 (m, 2H); 7.43-7.38 (m, 1H); 7.07-7.02 (m, 1H) 2.74 (d, J=6.6 Hz, 2H); 1.30-1.18 (m, 1H); 0.70-0.64 (m. 2H); 0.28-0.25 (m, 2H).

ESMS calculated for (C₁₉H₁₅Cl₂N₃O₂): 387.05; Found: 410.0 (M+Na)⁺.

### EXAMPLE 37

### Preparation of Compound 37: 2-(3-methyl-indolizin-1-yl)-2-oxo-N-(3,5-dichloro-pyridin-4-yl)-acetamide

Compound 37 was prepared in a manner similar to that described in Example 1.

¹H NMR (300 MHz, DMSO-D₆), δ (ppm): 8.90 (s, 2H); 8.38 (d, J=6.9 Hz, 1H); 7.85 (d, J=9.0 Hz, 1H); 7.11-7.32 (m, 4H); 2.50 (s, 3H).

ESMS calculated for (C₁₁H₁₀NO₂): 347.1; found: 346.1 (M−H)⁻.

### EXAMPLE 38

### Preparation of Compound 38: 2-[3-(4-cyano-benzyl)-indolizin-1-yl]-2-oxo-N-(3,5-dichloro-1-oxy-pyridin-4-yl)-acetamide

Compound 38 was prepared in a manner similar to that described in Example 1.

¹H NMR (300 MHz, DMSO-D₆) δ (ppm): 9.74 (br, s, 1H); 8.65 (d, J=9 Hz, 2H); 8.20 (d, J=7.2 Hz, 2H); 8.06 (s, 1H); 7.8-7.6 (m, 5H); 7.4 (m, 1H); 7.35 (d, J=7.8 Hz, 2H); 6.9 (m, 2H); 4.34 (s, 2H).

ESMS calculated for (C₂₃H₁₆N₄O₃): 396.12; found: 397.1 (M+H)⁺.

### EXAMPLE 39

### Preparation of Compound 39: 2-[3-(4-fluro-benzyl)-indolizin-1-yl]-2-oxo-N-(3,5-dichloro-1-oxy-pyridin-4-yl)-acetamide

Compound 39 was prepared in a manner similar to that described in Example 1.

¹H-NMR (CD₃COCD₃) δ (ppm): 8.41 (d, J=10, 1H); 8.22 (d, J=8, 1H); 7.43-6.95 (m, 9H); 4.36 (s, 2H).

ESMS calculated for (C₂₂H₁₄FCl₂N₃O₃): 457.04; found: 457.0.

### EXAMPLE 40

### In Vitro Assay (Inhibition of Human TNFα)

Reagents. Lipopolysaccharide (LPS, Serratia marscencens) was obtained from Sigma (St. Louis, Mo.). RPMI-1640 medium and fetal calf serum (FCS) were purchased from the ATCC (Manassas, Va.).

Human In Vitro Assay. Human peripheral blood cells (PBMC) were isolated by centrifugation using Ficoll-Paque (Pharmacia Biotech, Uppsala, Sweden) and suspended in RPMI-1640 medium supplemented with 10% FCS, 100 U/mL penicillin, and 100 μg/mL streptomycin. The cells were then plated in the wells of a 96-well plate at a concentration of 5×105 cells/well, and stimulated by adding LPS (1 μg/mL). Each test compound was dissolved in DMSO and added to the wells. The final DMSO concentration was adjusted to 0.25% in all cultures, including the compound-free control, and the concentrations of each test compound ranged from 0 to 10 μM. Cell-free supernatants were taken 18 h later for measurement of cytokines. Cell viability was assessed using the bioreduction of MTS [3-(4,5-dimethylthiazol-2-yl)-5-(3-carboxymethoxyphenyl)-2-(4-sulophenyl)-2H-tetrazolium] after 18 h and 48 h. Cell survival was estimated by determining the ratio of the absorbance in each of the compound-treated cultures to that in the compound-free control.

The supernatant was assayed for the amount of TNFα by using an ELISA assay with antihuman TNFα antibodies (Cell Sciences, Norwood, Mass.). The assay was carried out following the manufacturer's instructions.

Compounds 1-8, 11-32, 34, and 36-38 were tested. Unexpectedly, 32 of the compounds tested showed IC₅₀ values lower than 5 μM, and 5 showed IC₅₀ values of 10 nM or lower. Even at the highest concentration (10 uM), none of the test compounds affected cell viability after 48 h.

### EXAMPLE 41

### In Vitro Assay (Inhibition of PDE4)

PDE4 was prepared from U937 human monocytic cells according to the method of Tenor et al. (Clin Exp Allegy (1995) 25:625-633). Briefly, U937 cells were homogenized in a mixture of pH 7.4 containing 10 mM Hepes, 1 mM b-mercaptoethanol, 1 mM MgCl₂, 1 mM EGTA, 137 mM NaCl, 2.7 mM KCl, 1.5 mM KH₂PO₄, 8.1 mM Na₂HPO₄, 5 μM pepstain A, 10 μM leupeptin, 50 μM PMSF, 10 μM soybean trypsin inhibitor, and 2 mM benzamindine. The homogenate was centrifuged at 200,000×g for 30 min. PDE4 activity in the supernatant was assayed in a 200 μl reaction containing 40 mM Tris-HCl, pH 7.5, 23 nM [³H]-adenosine 3′,5′ cyclic monophosphate (cAMP), 8.3 mM MgCl₂, 1.7 mM EGTA, 0.25% DMSO, and a testing compound. The assay mixture was incubated at 37° C. for 30 min and the reaction was terminated by the addition of 100 μl of yttrium silicate SPA beads (Amersham Pharmacia Biotech, Piscataway, N.J.) suspended in 18 mM ZnSO₄. The assay mixture was rotated for 3 min to ensure the binding of [³H]-5′adenosine monophosphate to the beads. Finally, the beads was spun down, washed twice with 6 mM ZnSO₄, resuspended in 100 μl of 0.1 N NaOH, and then counted for radioactivity in a liquid scintillation counter.

Compounds 1, 2-4,8-10, 11, 15-23, 28-32, 35, 36, and 39 were tested. All tested compounds showed IC₅₀ values lower than 5 μM, and 4 of them showed IC₅₀ values lower than 100 nM.

### EXAMPLE 42

### In Vivo Assay (Edema)

Male Sprague-Dawley rats (Charles River Laboratories, Wilmington, Mass.), weighing 120-180 gram, were used throughout this study. A 1% (wt/vol) solution of lambda carrageenan (Sigma, St. Louis, Mo.) in saline was prepared freshly for each experiment. Compound 16 was formulated in 10% DMSO and 18% cremophore for intravenous administration and formulated in 1% methylcellulose (MC) (mol. Wt. 5000) for oral administration. Groups of 5 male rats were selected for study. Before carrageenan injection, compound 16 was intravenously or orally administered. Thirty minutes later, the rats were lightly anesthetized and 0.1 mL of carrageenan solution was injected by a subplantar route into the right hind paw. Paw volumes before and after carrageenan challenge were measured using hydroplethysmograph (Socrel, Varese, Italy), and the increase in volume caused by the irritant was determined after subtracting the volume of the paw before injection. Up to 69% inhibition of paw volume increase was achieved.

### EXAMPLE 43

### In Vivo Assay (Septic Shock)

Septic shock was elicited by two consecutive injection of E. Coli 055: B5 LPS in 9 week old female Balb/c mice (Taconic Farms, Germantown, N.Y.). The test compounds were formulated in 10% DMSO and 18% cremophore. Groups of 5 female mice weighing 19-20 gram were selected for study. E. Coli 055:B5 was reconstituted in phosphate buffered saline (PBS).

The priming injection was given in the footpad with 1.5 μg LPS per mouse. 24 h later the test compounds were intravenously or orally administered, followed by a challenge of 250 μg of LPS injected intravenously. Mortality was monitored after 24, 48 and 72 hours.

Compounds 11, 16, 17, 19, and 20 were tested. Mice in the vehicle control group were all dead after 24 hours. Mice treated with the tested compounds showed a higher survival rate. Indeed, all mice in groups treated with compounds 16 and 20 survived after 72 hours.

### EXAMPLE 44

### In Vivo Assay (Crohn's Disease)

Wistar derived male or female rats (Charles River Laboratories, Wilmington, Mass.) weighing 200±20 g and fasted for 24 hours, were used. Distal colitis was induced by intra-colonic instillation of 2,4-dinitrobenzene sulfonic acid (DNBS, 25 mg in 0.5 mL 30% ethanol solution) after which 2 mL of air was gently injected through the cannula to ensure that the solution remained in the colon. Compound 20 was administered orally 24 hours and 2 hours daily for 5 days before the DNBS instillation. One control group was treated with vehicle alone, while the other was treated with both DNBS and vehicle. The animals were sacrificed 24 hours after the final administration and the colons were removed and weighed. The colon-to-body weight ratio was then calculated for each animal. The increase in ratio of DNBS+vehicle control group relative to vehicle control group was used as a base for comparison. In the treated group, a 45% deduction in the inflammatory response was observed.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

For example, the indolizine compounds of this invention can also be used to treat TNFα- or PDE4-related diseases other than inflammatory disorders. Further, these compounds can bring about therapeutic effects either via inhibition of TNFα or PDE4, or via any other mechanisms. Additional utilities include their applications in screening, research, and diagnosis.

What is claimed is:

1. A compound of the formula: 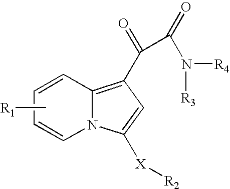
wherein
R₁ is H, lower alkyl, lower alkoxy, OH, F, Cl, Br, I, NO₂, or CN;
R₂ is alkyl optionally substituted with lower alkoxy, OH, CN, F, Cl, Br, I, NO₂, NH₂, C(O)NH₂, CO₂H, or CO₂R′; or aryl optionally substituted with lower alkyl, lower alkoxy, OH, CN, F, Cl, Br, I, NO₂, NH₂, or C(O)NH₂, CO₂H, or CO₂R′;
R₃ is H or lower alkyl;
R₄ is N-oxy pyridyl or pyridyl optionally substituted with F, Cl, Br, or I; and
X is C(R′R″), N(R′), O, S, S(O), S(O)₂, C(O), C(O)—N(R′), N(R′)—C(O), or deleted; in which each of R′ and R″, independently, is H, or alkyl optionally substituted with lower alkoxy, OH, CN, F, Cl, Br, I, NO₂, NH₂, or C(O)NH₂.

2. The compound of claim 1, wherein
R₁ is H, OH, F, or Cl;
R₂ is phenyl optionally p-substituted with lower alkoxy, OH, CN, F, Cl, Br, I, NO₂, NH₂, C(O)NH₂, CO₂H, or CO₂R′;
R₃ is H;
R₄ is N-oxy p-pyridyl or p-pyridyl optionally substituted with F, Cl, Br, or I; and
X is CH₂.

3. The compound of claim 1, wherein
R₁ is H, OH, F, or Cl;
R₂ is phenyl optionally p-substituted with lower alkoxy, OH, CN, F, Cl, Br, I, NO₂, NH₂, C(O)NH₂, CO₂H, or CO₂R′;
R₃ is H;
R₄ is o-pyridyl optionally substituted with F, Cl, Br, or I; and
X is CH₂.

4. The compound of claim 1, wherein
R₁ is H, OH, F, or Cl;
R₂ is phenyl optionally p-substituted with lower alkoxy, OH, CN, F, Cl, Br, I, NO₂, NH₂, C(O)NH₂, CO₂H, or CO₂R′;
R₃ is H;
R₄ is m-pyridyl optionally substituted with F, Cl, Br, or I; and
X is CH₂.

5. The compound of claim 2, wherein R₁ is H.

6. The compound of claim 2, wherein R₁ is OH.

7. The compound of claim 2, wherein R₁ is F.

8. The compound of claim 3, wherein R₁ is H.

9. The compound of claim 4, wherein R₁ is H.

10. The compound of claim 4, wherein R₁ is OH or F.

11. The compound of claim 5, wherein R₂ is phenyl p-substituted with CN, F, or Cl.

12. The compound of claim 6, wherein R₂ is phenyl p-substituted with F.

13. The compound of claim 7, wherein R₂ is phenyl p-substituted with F or OH.

14. The compound of claim 8, wherein R₂ is phenyl p-substituted with CN and R₄ is pyridyl.

15. The compound of claim 8, wherein R₂ is phenyl or phenyl p-substituted with Cl or F.

16. The compound of claim 9, wherein R₂ is phenyl or phenyl p-substituted with CN, F, Cl, NO₂, NH₂, or C(O)NH₂.

17. The compound of claim 10, wherein R₂ is phenyl or phenyl p-substituted with CN, F, Cl, NO₂, NH₂, or C(O)NH₂.

18. The compound of claim 11, wherein R₄ is pyridyl di-substituted with Cl at 2- and 6-positions.

19. The compound of claim 12, wherein R₄ is pyridyl di-substituted with Cl at 2- and 6-positions.

20. A pharmaceutical composition comprising
a compound of the formula: 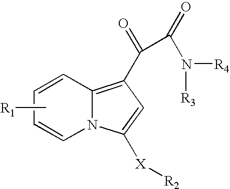
wherein
R₁ is H, lower alkyl, lower alkoxy, OH, F, Cl, Br, I, NO₂, or CN;
R₂ is alkyl optionally substituted with lower alkoxy, OH, CN, F, Cl, Br, I, NO₂, NH₂, C(O)NH₂, CO₂H, or CO₂R′; or aryl optionally substituted with lower alkyl, lower alkoxy, OH, CN, F, Cl, Br, I, NO₂, NH₂, C(O)NH₂, CO₂H, or CO₂R′;
R₃ is H or lower alkyl;
R₄ is N-oxy pyridyl or pyridyl optionally substituted with F, Cl, Br, or I; and
X is C(R′R″), N(R′), O, S, S(O), S(O)₂, C(O), C(O)—N(R′), N(R′)—C(O), or deleted; in which each of R′ and R″, independently, is H, or alkyl optionally substituted with lower alkoxy, OH, CN, F, Cl, Br, I, NO₂, NH₂, or C(O)NH₂; and
a pharmaceutically acceptable carrier.

21. The composition of claim 20, wherein
R₁ is H, OH, F, or Cl;
R₂ is phenyl optionally p-substituted with lower alkoxy, OH, CN, F, Cl, Br, I, NO₂, NH₂, C(O)NH₂, CO₂H, or CO₂R′;
R₃ is H;
R₄ is N-oxy p-pyridyl or p-pyridyl optionally substituted with F, Cl, Br, or I; and
X is CH₂.

22. The composition of claim 20, wherein
R₁ is H, OH, F, or Cl;
R₂ is phenyl optionally p-substituted with lower alkoxy, OH, CN, F, Cl, Br, I, NO₂, NH₂, C(O)NH₂, CO₂H, or CO₂R′;
R₃ is H;
R₄ is o-pyridyl optionally substituted with F, Cl, Br, or I; and
X is CH₂.

23. The composition of claim 20, wherein
R₁ is H, OH, F, or Cl;
R₂ is phenyl optionally p-substituted with lower alkoxy, OH, CN, F, Cl, Br, I, NO₂, NH₂, C(O)NH₂, CO₂H, or CO₂R′;
R₃ is H;
R₄ is m-pyridyl optionally substituted with F, Cl, Br, or I; and
X is CH₂.

24. The composition of claim 21, wherein R₁ is H and R₂ is phenyl p-substituted with CN, F, or Cl.

25. The composition of claim 21, wherein R₁ is OH and R₂ is phenyl p-substituted with F.

26. The composition of claim 22, wherein R₁ is H; R₂ is phenyl or phenyl p-substituted with CN; and R₄ is pyridyl.

27. The composition of claim 23, wherein R₁ is H; R₂ is phenyl p-substituted with CN, F, Cl, NO₂, NH₂, or C(O)NH₂; and R₄ is pyridyl.

28. A method for treating an inflammatory disorder, comprising administering to a subject in need thereof an effective amount of the compound of the formula: 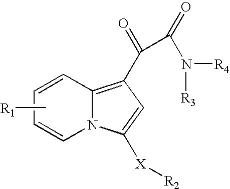
wherein
R₁ is H, lower alkyl, lower alkoxy, OH, F, Cl, Br, I, NO₂, or CN;
R₂ is alkyl optionally substituted with lower alkoxy, OH, CN, F, Cl, Br, I, NO₂, NH₂, C(O)NH₂, CO₂H, or CO₂R′; or aryl optionally substituted with lower alkyl, lower alkoxy, OH, CN, F, Cl, Br, I, NO₂, NH₂, C(O)NH₂, CO₂H, or CO₂R′;
R₃ is H or lower alkyl;
R₄ is N-oxy pyridyl or pyridyl optionally substituted with F, Cl, Br, or I; and
X is C(R′R″), N(R′), O, S, S(O), S(O)₂, C(O), C(O)—N(R′), N(R′)—C(O), or deleted; in which each of R′ and R″, independently, is H, or alkyl optionally substituted with lower alkoxy, OH, CN, F, Cl, Br, I, NO₂, NH₂, or C(O)NH₂.

29. The method of claim 28, wherein
R₁ is H, OH, F, or Cl;
R₂ is phenyl optionally p-substituted with lower alkoxy, OH, CN, F, Cl, Br, I, NO₂, NH₂, C(O)NH₂, CO₂H, or CO₂R′;
R₃ is H;
R₄ is N-oxy p-pyridyl or p-pyridyl optionally substituted with F, Cl, Br, or I; and
X is CH₂.

30. The method of claim 28, wherein
R₁ is H, OH, F, or Cl;
R₂ is phenyl optionally p-substituted with lower alkoxy, OH, CN, F, Cl, Br, I, NO₂, NH₂, C(O)NH₂, CO₂H, or CO₂R′;
R₃ is H;
R₄ is o-pyridyl optionally substituted with F, Cl, Br, or I; and
X is CH₂.

31. The method of claim 28, wherein
R₁ is H, OH, or Cl;
R₂ is phenyl optionally p-substituted with lower alkoxy, OH, CN, F, Cl, Br, I, NO₂, NH₂, C(O)NH₂, CO₂H, or CO₂R′;
R₃ is H;
R₄ is m-pyridyl optionally substituted with F, Cl, Br, or I; and
X is CH₂.

32. The method of claim 29, wherein R is —H and R₂ is phenyl p-substituted with CN, F, or Cl.

33. The method of claim 29, wherein R is OH and R₂ is phenyl p-substituted with F.

34. The method of claim 30, wherein R₁ is H; R₂ is phenyl or phenyl p-substituted with CN; and R₄ is pyridyl.

35. The method of claim 31, wherein R₁ is —H; R₂ is phenyl p-substituted with CN, F, Cl, NO₂, NH₂, or C(O)NH₂; and R₄ is pyridyl.
